(19) United States
(12) Patent Application Publication
(10) Pub. No.: US 2007/0010747 A1
(43) Pub. Date: Jan. 11, 2007

(54) Methods and systems for acquiring ultrasound image data

(76) Inventors: Thomas James Sabourin, Milwaukee, WI (US); Michelle Ganiere Angle, Muskego, WI (US); Robert B. Thompson, Burnaby (CA)

Correspondence Address: THE SMALL PATENT LAW GROUP LLP, 611 OLIVE STREET, SUITE 1611, ST. LOUIS, MO, 63101, US

(21) Appl. No.: 11/138,199

(22) Filed: May 26, 2005

(51) Int. Cl. A61B 8/00

(52) U.S. Cl. 600/453

(57) ABSTRACT

Methods and systems for acquiring ultrasound image information are provided. The method includes receiving compounding image information from an ultrasound system, receiving color flow image information from the ultrasound system, and processing the received compounding image information and color flow image information to generate a compounded ultrasound image in combination with color flow imaging.

### BACKGROUND OF THE INVENTION

This invention relates generally to ultrasound systems and, more particularly, to methods and systems for acquiring ultrasound image data.

Ultrasound systems typically include ultrasound scanning devices, such as, ultrasound probes having different transducers that allow for performing various different ultrasound scans (e.g., different imaging of a volume or body). Additionally, different modes of operation are typically available, such as, for example, amplitude mode (A-mode), brightness mode (B-mode), etc.

Further, different methods for improving the quality (e.g., resolution) of scanned images are known. For example, spatial compounding may be provided by an ultrasound system. Specifically, spatial compounding combines frames of images acquired at different geometries (e.g., images at different angles on a liner probe) into a single composite image. This compounded image provides improved image quality relative to conventional or non-compounded scanning by improving the contrast resolution. However, because of the manner in which the spatially compounded images are acquired, and in particular, the manner in which the necessary data for spatial compounding is acquired, as well as the processing demands necessary to produce real time images, it is difficult to use spatial compounding in combination with other modes of operation. For example, it is difficult to use spatial compounding with color flow imaging, power Doppler or other two-dimensional (2D) image modes that depict, for example, blood velocity information.

### BRIEF DESCRIPTION OF THE INVENTION

In one embodiment, a method for performing ultrasound imaging is provided. The method includes receiving compounding image information from an ultrasound system, receiving color flow image information from the ultrasound system, and processing the received compounding image information and color flow image information to generate a compounded ultrasound image in combination with color flow imaging.

In another embodiment, an acquisition system for acquiring ultrasound information in an ultrasound system is provided. The acquisition system includes a data acquisition component for acquiring color flow imaging information and compounding image information, a memory for storing frames of acquired color flow imaging information and compounding image information, a compound processing component for processing the frames of compounding image information, and a non-compound processing component for processing the frames of compounding information. The acquisition system also includes a switch for selecting frames from the memory to be processed by at least one of the compound processing component and non-compound processing component and a color flow processing component for processing the frames of color flow image information. The acquisition system further includes a display for displaying a plurality of images from the processed acquired information based upon a user input, the images displayed include at least one of a compounded image and a non-compounded image, and at least one of a compounded image with color flow overlaid and a non-compounded image with color flow overlaid.

### BRIEF DESCRIPTION OF THE DRAWINGS

FIG. 1 is a block diagram of an ultrasound system in accordance with an exemplary embodiment of the present invention.

FIG. 2 is a diagram illustrating acquiring an image of an object using the system of FIG. 1 in accordance with an exemplary embodiment of the present invention.

FIG. 3 is a diagram illustrating spatial compounding in accordance with an exemplary embodiment of the present invention.

FIG. 4 is a block diagram illustrating an acquisition system in accordance with an exemplary embodiment of the present invention.

FIG. 5 is a block diagram showing different compounding frame perspectives that may be acquired by a probe of an ultrasound system in accordance with an exemplary embodiment of the present invention.

FIG. 6 is a block diagram of an acquisition sequence in accordance with an exemplary embodiment of the present invention.

FIG. 7 is a block diagram of an acquisition sequence in accordance with another exemplary embodiment of the present invention.

FIG. 8 is a block diagram of an acquisition sequence in accordance with another exemplary embodiment of the present invention.

FIG. 9 is a block diagram of an acquisition sequence in accordance with another exemplary embodiment of the present invention.

FIG. 10 is a block diagram of an acquisition sequence in accordance with another exemplary embodiment of the present invention.

### DETAILED DESCRIPTION OF THE INVENTION

Exemplary embodiments of ultrasound systems and methods for acquiring and combining ultrasound image data/information are described in detail below. In particular, a detailed description of exemplary ultrasound systems will first be provided followed by a detailed description of various embodiments of methods and systems for acquiring ultrasound data/information. It should be noted that the terms data and information are used interchangeably herein.

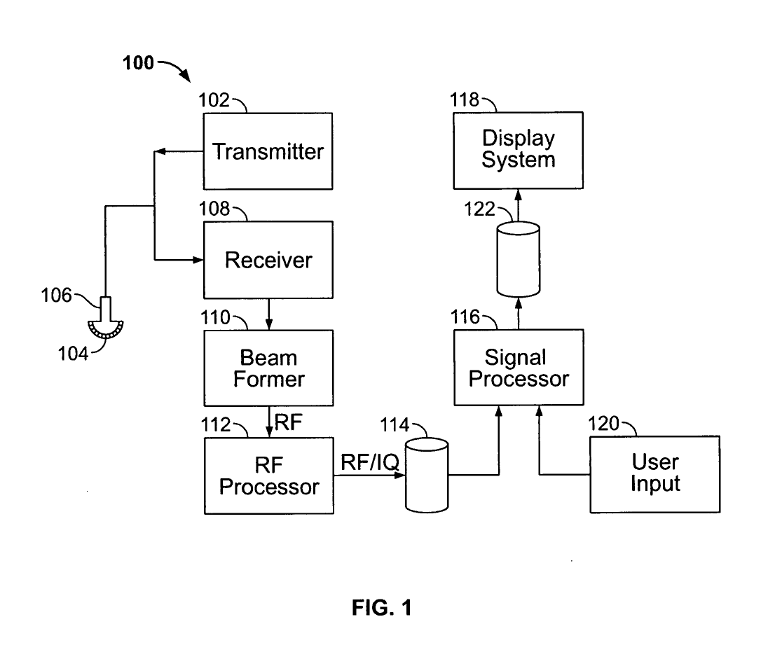

FIG. 1 illustrates a block diagram of an exemplary embodiment of an ultrasound system 100 that may be used, for example, to acquire and process ultrasonic images. The ultrasound system 100 includes a transmitter 102 that drives an array of elements 104 (e.g., piezoelectric crystals) within or formed as part of a transducer 106 to emit pulsed ultrasonic signals into a body or volume. A variety of geometries may be used and one or more transducers 106 may be provided as part of a probe (not shown). The pulsed ultrasonic signals are back-scattered from density interfaces and/or structures, for example, in a body, like blood cells or muscular tissue, to produce echoes that return to the elements 104. The echoes are received by a receiver 108 and provided to a beamformer 110. The beamformer performs beamforming on the received echoes and outputs an RF signal. The RF signal is then processed by an RF processor 112. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and prepare frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information is displayed on the display system 118 at a slower frame-rate. An image buffer 122 may be included for storing processed frames of acquired ultrasound information that are not scheduled to be displayed immediately. In an exemplary embodiment, the image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of the ultrasound system 100. The user input device 120 may be any suitable device and/or user interface for receiving user inputs to control, for example, the type of scan or type of transducer to be used in a scan.

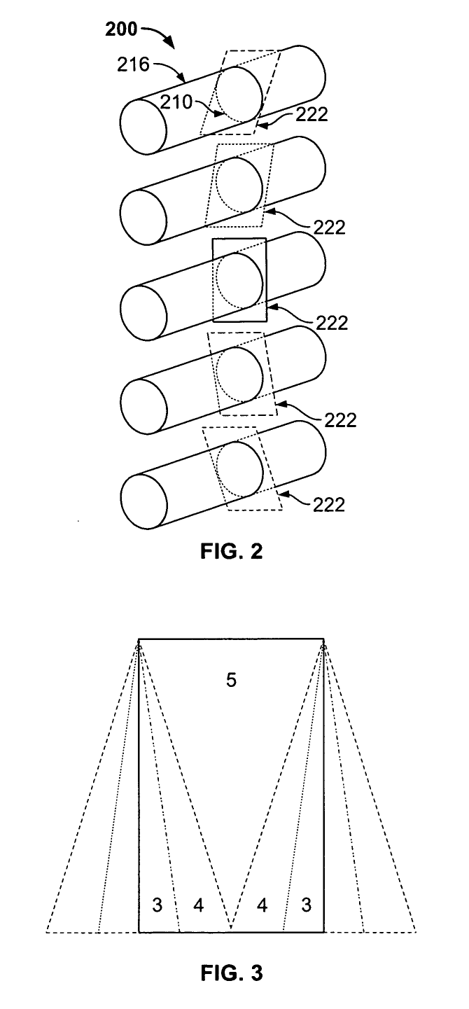

FIG. 2 illustrates the acquisition of an image of an object 200. The acquisition may be performed, for example, using the ultrasound system 100. It should be noted that although the object imaged is a volume, different images may be acquired, such as, for example, 2D images. An image of the object 200 is defined by a plurality of cross-sections 210 acquired by a plurality of compounding frames 222 to generate an imaged volume 216 as described in more detail herein.

Further, and as shown in FIG. 3, spatial compounding may be provided and includes combining frames of B-mode data from multiple co-planar views of the same anatomical region into one frame of data for display. Frames are acquired in a repeating manner from different lines-of-sight. FIG. 2 illustrates the same cross-sectional slice 210 of a volume 216 being interrogated from five different directions corresponding to the frames 222. As each frame 222 is acquired, the frame is combined with the previous four frames to produce an output frame in the geometric space of the un-steered frame. FIG. 3 illustrates different regions of the output frame that are comprised of overlapping regions of the input frames. As shown, the displayed output frame has the geometry of the un-steered frame. In the example, the top portion of the output frame is formed by combining data from all five directions (the two steered to the left, two to the right, and the single un-steered frame). The remainder of the output frame is a result of the combination of three or four frames, depending on the number of frames that overlap the region.

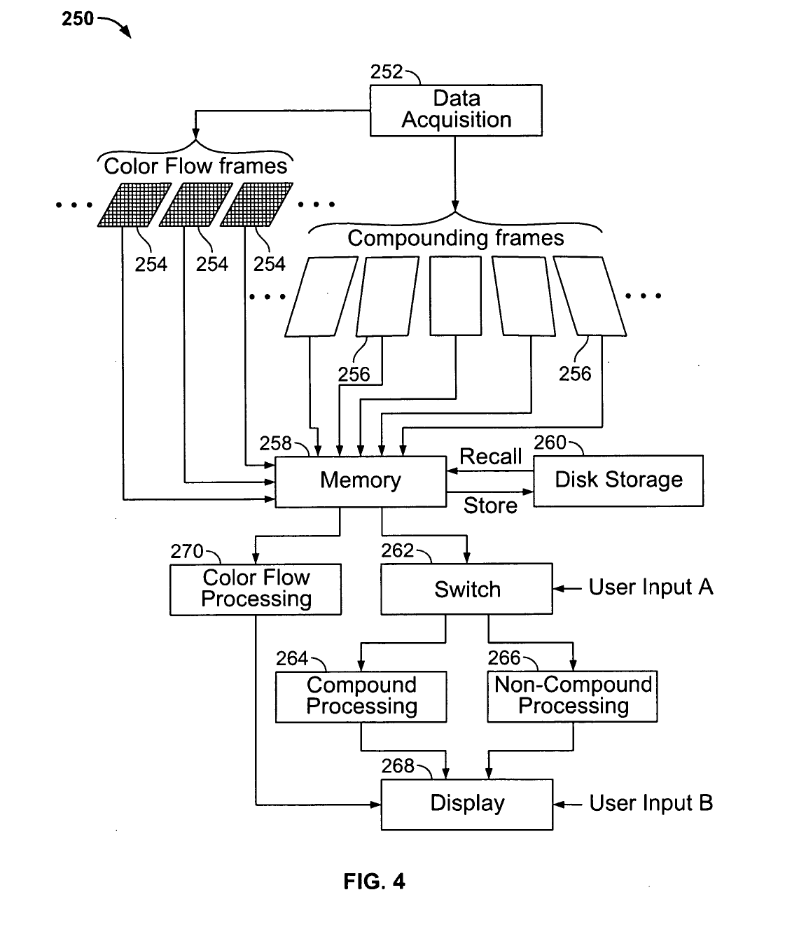

FIG. 4 illustrates a block diagram of an acquisition system 250 in accordance with an exemplary embodiment of the present invention that may be used in connection with the ultrasound systems 100. Specifically, the acquisition system 250 includes a data acquisition component 252 which may include, for example, the transducer 106, the transmitter 102, and the receiver 108 (all shown in FIGS. 1 and 2). As should be appreciated, the data acquisition component 252 may acquire ultrasound image data/information in different modes of operation and using different transducers or probes. For example, and as shown in FIG. 4, color flow imaging may be performed with color flow frames 254 acquired and compound imaging may be performed with compounding frames 256 acquired, with the frames acquired using the data acquisition component 252.

The acquired frames of images are stored in a memory 258, which in one exemplary embodiment is a short term memory (e.g., random access memory). A long term memory such as, for example, a disc storage 260 may be provided for storing selected or desired images for later recall and display. A switch 262 is also provided and may be operated by a user using the user input device 120 (shown in FIG. 1). The switch 262 allows the user to select information (e.g., image frames) in memory 258 to be provided to a compound processing component 264, a non-compound processing component 266, or both, to process acquired image data (e.g., frames). The processed image data is then displayed on a display 268 and may include one or both of a compounded or a non-compounded image based upon the acquired frames from the data acquisition component 252. Additionally, a color flow processing component 270 is connected to the memory 258 for processing the color flow frames for display on the display 268 in addition or in combination with the compounded or non-compounded images from the compound processing component 264 and non-compound processing component 266. A user input may be provided via the user input device 120 (shown in FIG. 1) to select, for example, the type or configuration of images to be displayed and as described herein. It should also be noted that the display 268 may be any suitable display including the display system 118 (shown in FIG. 1).

It further should be noted that the component parts of the acquisition system 250 may be constructed and/or provided as desired or needed, for example, based upon the particular ultrasound system. Thus, different component parts may implemented to perform the various operations and functions as described herein.

In operation, using the acquisition system 250 and the methods described below, compounded and/or non-compounded images that may have color flow combined therewith (e.g. overlaid) may be provided as desired or needed. For example, the display 268 may display two separate images such as a compounded image on one portion of the display 268 and a compounded image with overlaid color flow on another portion of the display 268.

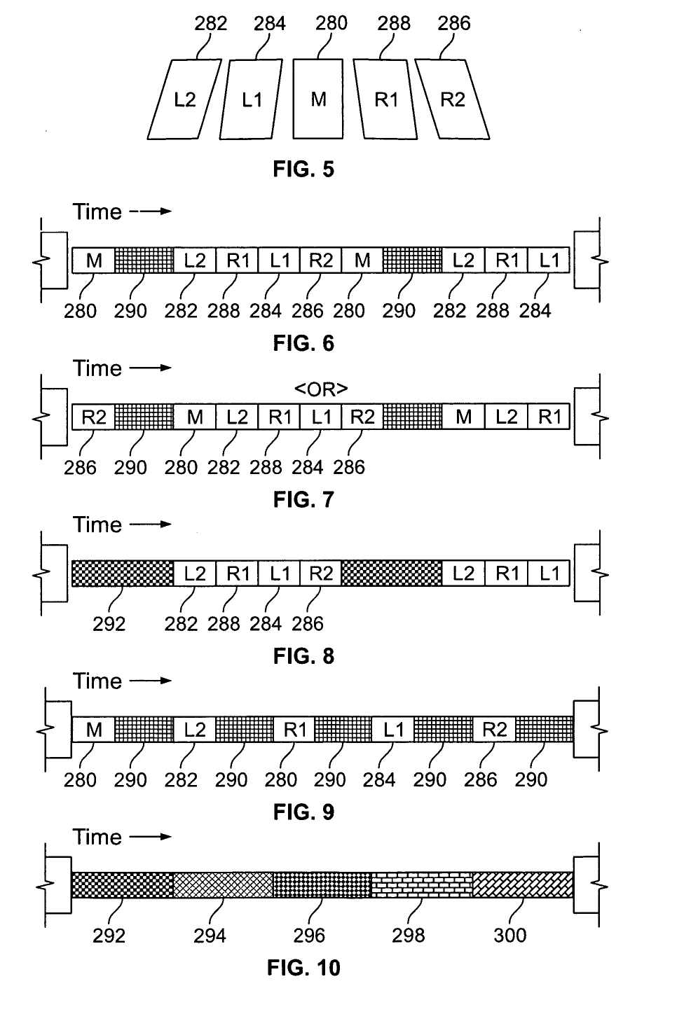

With respect to the acquisition of ultrasound image data/information using the data acquisition component 252, compounding frames and color flow frames are acquired during a single scan. As shown in FIG. 5, the compounding frames may be acquired at different angles with respect to the same point and/or region of interest to be scanned. Specifically, B-mode firings for a single frame are shown wherein a middle (M) firing 280 is provided with no angle and a left 2 (L2) firing 282 and a left 1 (L1) firing 284 are provided angled (e.g., to the left relative to the region of interest) with respect to the middle firing 280. For example, the L1 firing 284 may be provided at fifteen degrees and the L2 firing 282 may be provided at thirty degrees, respectively, relative to the middle firing 280. Similarly, a right 2 (R2) firing 286 and a right 1 (R1) firing 288 likewise may be provided at an angle with respect to the middle firing 280. As should be appreciated, these different firings may be provided by either mechanically steering or directing the transducer 106 (e.g., moving scan head in a probe) and/or electrically (e.g., using a phased array). When reference is made herein to a firing, this generally means activating an ultrasound system to acquire images, such as, for example, driving an array of elements as part of a transducer to emit pulsed ultrasonic signals into a body or volume.

As shown in FIGS. 6 through 10, various embodiments of the present invention provide acquisition sequences using the acquisition system 250 including the data acquisition component 252 to acquire color flow image data (e.g., color flow frames 254) and compounded image data (e.g., compounding frames 256) during a single scan, which may be in real time, and for a given object, such as the same anatomy. The acquisition sequences allow, for example, a user to scan with compounding turned on, but also having a non-compound version of the image displayed in real time next to the compounded image with the option to overlay anatomical information and color flow image information on either or both of the images displayed on the display.

In particular, the data acquisition component 252 acquires and stores color flow frames 254 and compounding frames 256 in memory 258 as individual frames in one exemplary embodiment. Specifically, with respect to the acquisition of image information as shown in one exemplary embodiment in FIG. 6, one frame of color flow data per series or set of compounding frames is acquired either before or after a frame of B-mode data for a non-steered compounding frame in a non-interleaved manner. For example, a complete set of firings are performed to generate the entire color flow frame before switching to B-mode firings. As shown in FIG. 6, the following acquisition sequence or firing sequence is provided:

1. M firing 280

2. Single frame color flow firing 290

3. L2 firing 282

4. R1 firing 288

5. L1 firing 284

6. R2 firing 286

This sequence may be repeated as desired and needed, for example, to provide a real-time or live display of an imaged anatomy. For example, for compounded frames, the last five frames acquired are combined and displayed (in the case of compounding by five different steering directions). For example, assume frames start numbering from zero and count up while scanning. Assume compounding five angles. After acquiring frames 0 to 4, one output frame for display is outputted. Then frame 5 is acquired and the display updated with the combination of frames 1, 2, 3, 4 and 5. Frame 6 is then acquired and the display updated with the combination of frames 2, 3, 4, 5 and 6. The process continues accordingly.

In another exemplary embodiment shown in FIG. 7, and similar to the acquisition sequence shown in FIG. 6, the following acquisition sequence or firing sequence is provided:

1. R2 firing 286

2. Single frame color flow firing 290

3. M firing 280

4. L2 firing 282

5. R1 firing 288

6. L1 firing 284

7. R2 firing 286

Again, this sequence may be repeated as desired or needed.

In another exemplary embodiment shown in FIG. 8, the acquisition sequence is similar to the one shown in FIGS. 6 and 7, but with color flow firings interleaved with B-mode firings for non-steered frames. Specifically, the following acquisition sequence or firing sequence is provided:

1. Interleaved firing 292

2. L2 firing 282

3. R1 firing 288

4. L1 firing 284

5. R2 firing 286

Again, this sequence may be repeated as desired or needed.

It should be noted that the interleaved firing 292 is an interleaved color flow firing for a single frame and a B-mode firing for single frame at a particular steering direction. In this exemplary embodiment, the interleaved firing 292 is a combination of an M firing 280 and a single frame color flow firing 290. It should be noted that the interleaved firings may be provided as desired or needed (e.g., different combinations).

In another exemplary embodiment of an acquisition sequence as shown in FIG. 9, one frame of color flow data per individual compounding frame is acquired in a non-interleaved manner (e.g., a complete set of firings may be performed to generate the entire color flow frame is before switching to B-mode firings). Specifically, the following acquisition sequence or firing sequence is provided:

1. M firing 280

2. Single frame color flow firing 290

3. L2 firing 282

4. Single frame color flow firing 290

5. R1 firing 288

6. Single frame color flow firing 290

7. L1 firing 284

8. Single frame color flow firing 290

9. R2 firing 286

10. Color flow firing 290

Again, this sequence may be repeated as desired or needed.

In another exemplary embodiment of an acquisition sequence as shown in FIG. 10, a sequence similar to the one in FIG. 9 is provided, but with the color flow firings interleaved with B-mode firings for each steered compounding frame. Specifically, the following acquisition sequence or firing sequence is provided:

1. Interleaved firing 292
2. Interleaved firing 294
3. Interleaved firing 296
4. Interleaved firing 298
5. Interleaved firing 300
   Again, this sequence may be repeated as desired or needed.

In one exemplary embodiment, the interleaved firing 292 is an interleaved (i) M firing 280 and (ii) a single frame color flow firing 290. The interleaved firing 294 is an interleaved (i) L2 firing 282 and (ii) a single frame color flow firing 290. The interleaved firing 296 is an interleaved (i) R1 firing 288 and (ii) a single frame color flow firing 290. The interleaved firing 298 is an interleaved (i) L1 firing 284 and (ii) a single frame color flow firing 290. The interleaved firing 300 is an interleaved (i) R2 firing 286 and (ii) a single frame color flow firing 290. The interleaved firings may be provided as desired or needed (e.g., different combinations).

With respect to the interleaved firings, it should be noted that the combination of the color flow firings for a single frame and B-mode firings for a single frame may be provided in any manner as desired or needed. For example, and with reference to interleaved firing 292, if the M firing 280 and single frame color flow firing 290 vectors are interleaved with fifty lines in color and one hundred lines in B-mode, the firings may be interleaved at ten lines each at a time, that is, ten lines of B-mode followed by ten lines of color flow, followed by ten lines of B-mode, etc. However, other combinations are possible as desired or needed. For example, from a firing standpoint in color, for each displayed line, 4 to 16 vectors may be fired in one particular direction to make one color line for display. The number of vectors is referred to as packet size. If there are 50 color lines for the display and 100 B-mode lines and the packet size for the color flow vectors is 8, the firing may be provided as follows: 20 B lines, 80 color (10 lines times 8 in a packet), then 20 B, then 80 color, until the frame is completed. Depending on user settings for PRF, depth, and color ROI size, the packets of color data can be interleaved. For example, the ROI may be divided into two regions of 25 lines each (200 firings total for each section or 25 times 8). In some situations, the first firing may be collected in a packet for the first region and then the first firing in a packet for the second region before acquiring the second firing for the first region. Using “L” for line and “F” for firing, the sequence may be represented as L1F1, L26F1, L1F2, L26F2, L1F3, L26F3, . . . , L1F8, L26F8, then some number of B lines, then L2F1, L27F1, L2F2, L27F2, L2F3, L27F3, . . . , L2F8, L27F8.

Also it should be noted that the angles used for the firings 280-288 and for acquiring the compounding frames may be programmable or predetermined as desired or needed, for example, based upon the particular application or probe being used. Additionally, different numbers of frames may be compounded, such as, 3, 5, 7, 9 and/or any other number as desired or needed.

Thus, various embodiments of the present invention allow a user to view on a display without having to switch between modes of display or operation different combinations of compounded and non-compounded images that may include color flow imaging. Thus, various embodiments of the present invention provide for displaying on a single display the anatomical image quality improvements of spatial compounding with physiological information, such as, for example, blood flow.

While the invention has been described in terms of various specific embodiments, those skilled in the art will recognize that the invention can be practiced with modification within the spirit and scope of the claims.

What is claimed is:

1. A method for performing ultrasound imaging, said method comprising:
receiving compounding image information from an ultrasound system;
receiving color flow image information from the ultrasound system; and
processing the received compounding image information and color flow image information to generate a compounded ultrasound image in combination with color flow imaging.

2. A method in accordance with claim 1 further comprising receiving power Doppler image information from the ultrasound system and wherein the processing comprises processing the received power Doppler image information to generate a compounded ultrasound image in combination with power Doppler imaging.

3. A method in accordance with claim 1 further comprising receiving two-dimensional blood image information from the ultrasound system and wherein the processing comprises processing the received two-dimensional blood image information to generate a compounded ultrasound image in combination with two-dimensional blood imaging.

4. A method in accordance with claim 1 further comprising displaying the combined compounded ultrasound image with color flow imaging on a display with a non-compounded ultrasound image, the images being of a same region of interest.

5. A method in accordance with claim 1 further comprising displaying the combined compounded ultrasound image with color flow imaging on a display with a combined non-compounded ultrasound image with color flow imaging, the images being of a same region of interest.

6. A method in accordance with claim 1 further comprising displaying the combined compounded ultrasound image with color flow imaging on a display with a compounded image, the images being of the same region of interest.

7. A method in accordance with claim 1 wherein the color flow image information is overlaid on the compounded ultrasound image.

8. A method in accordance with claim 1 further comprising receiving a user input to determine at least one ultrasound image to be displayed, the ultrasound images being at least one of a compounded and non-compounded image with at least one of the ultrasound images having color flow imaging overlaid thereon.

9. A method in accordance with claim 1 further comprising acquiring one frame of color flow image information for each set of frames of acquired compounding image information.

10. A method in accordance with claim 9 wherein at least one frame of the compounding image information is interleaved with the frame of color flow image information.

11. A method in accordance with claim 1 further comprising acquiring one frame of color flow image information for each frame of acquired compounding image information.

12. A method in accordance with claim 11 wherein one each of the frames of the color flow image information is interleaved with one each of the frames of acquired compounding image information.

13. A method in accordance with claim 1 further comprising acquiring the compounding information at different scan angles of a probe of the ultrasound system.

14. A method in accordance with claim 1 wherein the compounding image information is acquired using a B-mode operation of the ultrasound system.

15. A method for performing ultrasound imaging, said method comprising:
acquiring compounding image information with an ultrasound system;
acquiring color flow image information with the ultrasound system;
processing the color flow image information for display;
processing the compounding image information based on a user input; and
displaying a plurality of images from the processed acquired information based upon a user input, the images displayed being at least one of a compounded image and a non-compounded image, and at least one of a compounded image with color flow overlaid and a non-compounded image with color flow overlaid.

16. A method in accordance with claim 15 further comprising storing separately each frame of acquired compounding image information and each frame of acquired color flow image information.

17. A method in accordance with claim 15 wherein the acquiring comprises acquiring one frame of color flow image information for each set of frames of acquired compounding image information.

18. A method in accordance with claim 17 wherein at least one frame of the compounding image information is interleaved with the frame of color flow image information.

19. A method in accordance with claim 15 wherein the acquiring comprises acquiring one frame of color flow image information for each frame of acquired compounding image information.

20. A method in accordance with claim 19 wherein one each of the frames of the color flow image information is interleaved with one each of the frames of acquired compounding image information.

21. An acquisition system for acquiring ultrasound information in an ultrasound system, said acquisition system comprising:
a data acquisition component for acquiring color flow imaging information and compounding image information;
a memory for storing frames of acquired color flow imaging information and compounding image information;
a compound processing component for processing the frames of compounding image information;
a non-compound processing component for processing the frames of compounding information;
a switch for selecting frames from the memory to be processed by at least one of the compound processing component and non-compound processing component;
a color flow processing component for processing the frames of color flow image information; and
a display for displaying a plurality of images from the processed acquired information based upon a user input, the images displayed being at least one of a compounded image and a non-compounded image, and at least one of a compounded image with color flow overlaid and a non-compounded image with color flow overlaid.
